(19) United States
(12) Patent Application Publication
(10) Pub. No.: US 2021/0401360 A1
(43) Pub. Date: Dec. 30, 2021

(54) HAIR ANALYSING DEVICE

(71) Applicant: Henkel AG & Co. KGaA, Duesseldorf (DE)

(72) Inventors: DIANE METTEN, Hamburg (DE); THORSTEN KNAPPE, Schenefeld (DE); ANNA PULS, Winsen (Luhe) (DE); ROLF BAYERSDOERFER, Hamburg (DE); KATHARINA ROSCHER, Hamburg (DE)

(73) Assignee: Henkel AG & Co. KGaA, Duesseldorf (DE)

(21) Appl. No.: 16/471,532

(22) PCT Filed: Dec. 11, 2017
(86) PCT No.: PCT/EP2017/082238

§ 371 Date: Jun. 19, 2019

(30) Foreign Application Priority Data
Dec. 20, 2016 (DE) 10 2016 225 564.9

(51) Int. Cl. A61B 5/00; A61B 5/107; G01N 33/483; G16H 40/67; G06F 16/9535

(52) U.S. Cl. CPC A61B 5/448; G01N 2021/8444; A61B 5/1075; A61B 5/443; A61B 5/4872; A61B 5/4875; A61B 5/6832; A61B 5/0002; A61B 5/742; G01N 33/483; G16H 40/67; G06F 16/9535; A61B 2562/0271; A61B 2562/029; A61B 5/0082

(57) ABSTRACT

Hair analysing device, hair examination system and method for providing hair condition information. In various embodiments, a hair analysing device is provided. The hair analysing device can have a device body provided with an adhesive layer on at least one side of its surface and configured to be releasably fastened in or on a user's hair by employing the adhesive layer, at least one sensor arranged in or on the device body for detecting at least a sensor value, and an electronic circuit device arranged in or on the device body, wherein the electronic circuit device is coupled to the at least one sensor for receiving the at least one sensor value, and wherein the electronic circuit device is configured to provide the received at least one sensor value to the user.

### CROSS-REFERENCE TO RELATED APPLICATION

This application is a U.S. National-Stage entry under 35 U.S.C. § 371 based on International Application No. PCT/EP2017/082238, filed Dec. 11, 2017, which was published under PCT Article 21(2) and which claims priority to German Application No. 10 2016 225 564.9, filed Dec. 20, 2016, which are all hereby incorporated in their entirety by reference.

### TECHNICAL FIELD

Hair analysing device, hair examination system and method for providing hair condition information are provided herein.

### BACKGROUND

The present disclosure relates to a hair analysing device, a hair examination system and a method for providing a recommendation relating to hair of a user.

Consumers often know little about the condition of their hair and often take incorrect care and styling products that weigh down the hair or make it difficult to handle.

Adequate determination of an individual hair condition (also known as hair status) can be time-consuming and costly. Likewise, a cosmetically incorrect or faulty treatment due to lack of baseline data can be time-consuming and costly.

A self-measurement of the body's own data by employing so-called wearables (that is, sensors that can be worn on the body, either for a short time period or for a longer time period) is an increasing trend in wide populations. Conventional wearables used, however, are only suitable for detecting general health parameters, for example, temperature and/or pulse, but not for determining a hair condition.

### SUMMARY

Hair analysing devices, hair examination systems including the hair analysing devices, and methods of providing hair condition information are provided herein. In an embodiment, a hair analysing device includes a device body, at least one sensor arranged in or on the device body, and an electronic circuit device arranged in or on the device body. The device body includes an adhesive layer on a surface of at least one side. The device body is configured to be releasably fastened by employing the adhesive layer in or on the hair of a user. The at least one sensor is arranged in or on the device body for detecting at least one sensor value. The electronic circuit device is coupled to the at least one sensor for receiving the at least one sensor value. The electronic circuit device is configured to provide a received at least one sensor value to the user.

In another embodiment, the hair analysing device includes a device body, at least one sensor for detecting at least one sensor value, and an electronic circuit device arranged in or on the device body. The device body is configured to be arranged in or on the hair of a user, wherein the device body is thread-shaped or has a thread-shaped region. The sensor or at least one of the sensors is arranged in the thread-shaped device body or the thread-shaped region of the device body. The electronic circuit device is coupled to the at least one sensor for receiving the at least one sensor value. The electronic circuit device is configured to provide a received at least one sensor value to the user.

In another embodiment, a method of providing hair condition information includes arranging a hair analysing device according to claim 1 on hair of a user. At least one sensor value of the at least one sensor of the hair analysing device is detected. The at least one sensor value is provided to the user.

### BRIEF DESCRIPTION OF THE DRAWINGS

The present disclosure will hereinafter be described in conjunction with the following drawing figures, wherein like numerals denote like elements, and wherein:

FIG. 1A to 1D are schematic representations of hair analysing devices according to various embodiments;

FIG. 2A is a schematic representation of a hair analysing system according to various embodiments in connection with an external data processing device;

FIG. 2B is a schematic representation of a hair examination system according to various embodiments;

FIG. 3 is a schematic representation of an application of a hair examination system according to various embodiments;

FIG. 4 is a flow chart of a method of providing hair condition information according to various embodiments; and

FIG. 5 is a flow chart of a method of providing hair condition information according to various embodiments.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

In various embodiments, a device to be arranged on or in the hair (also referred to as a “hair wearable” or “hairable”) is provided, which can be equipped with sensor technology suitable for detecting cosmetic hair parameters, for example, sufficiently. Sensor technology here is understood to mean a total of one or more sensors.

In various embodiments, the sensor can be directed to both “internal” and “external” data. Expressed otherwise, the at least one sensor can be configured to detect one or more “internal” and/or one or more “external” sensor value(s).

The term “internal” data is understood to mean data on a cosmetic status, on a hair condition, for example, on hair health and/or on a scalp condition, for example, a scalp health of the consumer (for example, hair thickness, porosity of the hair surface, sebum content).

The term “external” data is understood to mean data relating to a consumer's living environment, such as UV exposure, temperature (level and fluctuation), air humidity, etc.

The hair analysing device can have at least one sensor in various embodiments. The at least one sensor can be arranged in or on the hair analysing device. At least in cases in which the sensor is arranged in the hair analysing device and the sensor value to be sensed requires it, the sensor can be in physical and/or optical contact with a surface of the device body.

The hair analysing device (the hairable) can have different configurations in various embodiments. The hairable can preferably be designed at least partially in a flexible type that is adaptable to a respective head shape.

In various embodiments, the hair analysing device can have films and/or foils which can be applied to the hair and possibly also to the scalp in various embodiments by employing an adhesive agent, for example, an adhesive or a Velcro surface. In various embodiments, the at least one sensor can be arranged in the film or the foil.

In various embodiments, the hair analysing device can have a sensor-equipped element for fixing to the hair, for example, a hair tie, a hair clip, a barrette, a headband, or the like.

In various embodiments, the hair analysing device can have at least one thread-shaped appendage, which can be suitable for being woven into the hair. The at least one thread-shaped appendage can be attached to a device body. At least a part of the at least one sensor can be arranged within the thread-shaped appendage. The circuit device, which can be coupled to the at least one sensor, can be arranged within the device body.

The hair analysing device can be designed thread-shaped in various embodiments. The at least one sensor and the circuit device coupled to the at least one sensor can be arranged in the thread-shaped hair analysing device.

In various embodiments, the thread-shaped hair analysing device can be designed, for example, in terms of length, thickness and flexibility, so that it can be interlaced into the hair.

In various embodiments, the thread-shaped hair analysing device can have sensor-equipped extensions for hair extension, and/or sensor-equipped, hair-like implants.

In various embodiments, data collected by employing the hair analysing device (that is, the determined at least one sensor value and/or data and/or recommendations determined thereon) can be provided.

In various embodiments, only the determined sensor values can be transmitted to a display device (for example, a smartphone or the like) and displayed.

In various embodiments, the sensor values can be examined in different ways (for example, analyzed and/or evaluated). In this case, the examination can refer in various embodiments to currently collected data (a current cosmetic condition (also referred to as cosmetic status)) and/or to a temporal change of the data (of the cosmetic status).

In various embodiments, the analysis can be performed either by the hair analysing device itself, for example, by employing the circuit device.

In various embodiments, for example, preferably, the data can be transferred on/to an external data processing device (also referred to as an external platform), for example, to a smartphone with app, to a cloud, etc. The examination of the data can be carried out after the data has been transferred to the external data processing device in this manner, for example, to determine a recommendation.

In various embodiments, the data evaluation can be the basis of a recommendation, for example, a product and/or method recommendation, for example, the recommendation can have so-called DOs (instructive recommendations) and DON'Ts (warning recommendations), for example, (as an example of a DO) “Take shampoo X and style your hair with mousse Y” or (as an example of a DON'T) “Do not wash yet, try dry shampoo today”.

In various embodiments, the consumer can be provided with current, personal data regarding the cosmetic condition of his hair. Detecting (also referred to as collecting) of this data by the hair analysing device can be done autonomously and continuously.

In various embodiments, autonomous data collection can release the consumer from a need to consult a specialist (for example, hairdresser and/or beautician) or an external cumbersome analyzing device.

A continuous data collection of hair health can allow, in various embodiments, a representation and review of the course and outcome of a cosmetic treatment.

In various embodiments, a matching of the cosmetic treatment with individualized consumer data can facilitate an iterative cosmetic treatment cycle, improve an outcome of the cosmetic treatment, and/or increase a user's motivation to continue the treatment.

In various embodiments, the hair analysing device, for example, the hair analysing device provided with the adhesive agent, can be designed so that it can be applicable even with short hair.

In various embodiments, the hair analysing device, for example, the hair analysing device provided with the adhesive agent, can be designed to be arrangeable such that it can be at least partially in contact with a scalp of the user. This can make it possible to determine a scalp condition of the user in addition to the hair condition.

In various embodiments, the hair analysing device can be designed such that individual, few and/or flat hairs can be arranged in a detection region of the at least one sensor. This can facilitate the detection of some sensor values, for example, of a hair thickness or the like.

In various embodiments, a user can be provided with information about a status of the hair (also referred to as a hair condition) which can further be used to determine an individual recommendation matched to the hair condition of the user, for example, a product recommendation (for example, for a hair care product, hair coloring and/or a hair styling product) and/or a care recommendation, for example, a care recommendation which relates to the hair and/or the scalp of the user.

In various embodiments, the hair analysing device can have an electronic circuit device and at least one sensor. The electronic circuit device can be coupled to the at least one sensor for receiving at least one sensor value. In various embodiments, the electronic circuit device can be configured, based on the received at least one sensor value, to determine and provide to the user at least one recommendation (for example, a product recommendation for a hair care product, a hair color product or a hair styling product (for example, a product for short or permanent hair shaping), or a treatment recommendation, for example, to protect hair from the sun, to wash or to wait for the washing).

In various embodiments, the recommendation can be determined directly by employing the hair analysing device, that is, the electronic circuit device can be configured to determine the recommendation itself (also referred to as directly). For example, the electronic circuit device can be or have a data processing device, for example, it can be equipped with a memory and a processor, for example, a microprocessor, which can be configured, for example, by programming, to receive the sensor data and either provide it directly to the user, or to use the sensor data to provide the recommendation. For example, the sensor data can be compared with a database, which may have been obtained empirically, for example. Recommendations can be assigned to a plurality of sensor data in the database. For an assignment of recommendations, the sensor data can be used individually, for example, a kind of sensor value taken by itself, for example, a hair thickness (for example, for a recommendation of a product and/or a hair treatment suitable in view of the one sensor value, for example, the hair thickness), and/or the sensor data can be used in combination, for example, two or more sensor values which are used in combination, for example, a hair thickness/hair color combination, which, in view of the determined sensor values, can be assigned at least one recommendation, for example, a product and/or hair treatment suitable for the present combination of hair thickness and hair color. An accuracy in determining the recommendation can be increased by using a plurality of sensor values for determining the at least one recommendation in various embodiments.

In various embodiments, the electronic circuit device can be configured to indirectly determine the recommendation, for example, product or treatment recommendation. For example, the electronic circuit device (for example, in addition to a memory and a processor, for example, a microprocessor) can be equipped with a data transfer device which can be configured to transmit the sensor data received by the electronic circuit device to an external data processing device, for example, a computer, for example, a cloud, by which, for example, as described above for determining the recommendation by employing the electronic circuit device, the recommendation can be determined in order to provide the recommendation, for example, by transferring to a recommendation and/or by transferring the recommendation back to the electronic circuit device (for example, by employing the data transmission device). In various embodiments, the data transmission can take place in several stages, for example, by first transmitting the sensor data from the circuit device to the display device (for example, a smartphone, a tablet or the like), and the display device transmits the sensor data to the external data processing device (for example, the cloud).

In various embodiments, providing the recommendation to the user can include providing by transmitting the recommendation to a display device and displaying the recommendation. In various embodiments, providing the recommendation to the user can include providing the recommendation by transmitting the recommendation to a display device and announcements, for example, by employing a speaker attached in/on the display device.

The transmission can be done in various embodiments by employing a wireless transmission device. The wireless transmission device can be part of the electronic circuit device, for example. The wireless transmission device can, in various embodiments, have a chip or tag which enables wireless data transmission, for example, by employing Bluetooth, WLAN, Zigbee, NFC, Wibree, Threald, WiMAX or the like.

In various embodiments, the display device can have a computer screen, a smartphone, a tablet, a smartwatch, an iPad, a laptop, or the like.

In various embodiments, a hair examination system can be formed by a hair analysing device and the display device.

As a variation on the above examples, in various embodiments, other combinations of two or more recommendations can be provided by employing the display device.

In various embodiments, the at least one sensor can have a hair thickness sensor. The hair thickness in various embodiments can have an optical sensor, for example, a sensor on which a surface region of the device body, with which hair can be in contact, is imaged, so that a thickness of the hair (for example, as it appears on the surface region) can be evaluated by employing an image evaluation of the sensor, and/or an ultrasonic sensor which is configured to emit in a known manner an ultrasonic signal and to examine a portion of the ultrasonic signal reflected from a structure to be measured (the hair) and to determine the thickness of the hair therefrom, and/or a laser, which is used for a known per se thickness measurement method, for example, for an interferometry and/or a triangulation method. The measured sensor values can, as described above, either be evaluated directly in the hair analysing device, for example, by employing the electronic circuit device, or indirectly evaluated by being transmitted to an external data processing device to be evaluated there, for example, as described above.

In various embodiments, the at least one sensor can have an air humidity sensor. The at least one air humidity sensor can be in physical contact with a surface of the device body, for example, having a measurement opening. The contact region (for example, the measurement opening) can be arranged, for example, on a side of the device body facing away from the user's head.

In various embodiments, the at least one sensor can have a UV radiation sensor. The at least one UV radiation sensor can be in optical contact with a surface of the device body, for example, on a side of the device body facing away from a user's head. Expressed otherwise, the hair analysing device can be made UV transparent between a light detection region (for example, a detector) and a surface of the hair analysing device.

In various embodiments, the at least one sensor can have a spectrometer and/or a camera for detecting an absorption feature of a substance to be detected, for example, a lipid (for example, a lipid which can be part of a user's cutaneous sebum), a cysteic acid, wherein a cysteic acid content can be used as a measure of hair damage), of water or the like.

In various embodiments, the at least one sensor can have a thermometer for detecting a hair temperature, a scalp temperature, and/or an ambient temperature of the user.

In various embodiments, the at least one sensor can have a camera. The image recorded by the sensor can be provided to the electronic circuit device, which can, for example, determine hair color, hair porosity, hair thickness, or other optically detectable hair properties directly or indirectly, by comparison with reference data, as described above. The determined hair property (hair color, hair porosity or the like) can be included in determining a recommendation, for example, a product or treatment recommendation.

In various embodiments, the at least one sensor can have other detection devices known in principle, which can be arranged in a hair analysing device due to a general increasing miniaturization of such devices.

In various embodiments, a hair analysing device is provided. The hair analysing device can have a device body provided with an adhesive layer on at least one side of its surface and configured to be releasably fastened in or on a user's hair by employing the adhesive layer, at least one sensor arranged in or on the device body for detecting at least a sensor value, and an electronic circuit device arranged in or on the device body, wherein the electronic circuit device is coupled to the at least one sensor for receiving the at least one sensor value, and wherein the electronic circuit device is configured to provide the received at least one sensor value to the user.

In various embodiments, a hair analysing device is provided. The hair analysing device can have a device body configured to be arranged in or on the hair of a user, wherein the device body is thread-shaped or has a thread-shaped region, at least one sensor for detecting at least one sensor value, wherein the sensor or at least one of the sensors is arranged in the thread-shaped device body or thread-shaped region of the device body, and an electronic circuit device arranged in or on the device body, wherein the electronic circuit device is coupled to the at least one sensor for receiving the at least one sensor value, and wherein the electronic circuit device is configured to provide the received at least one sensor value to the user.

In various embodiments, the electronic circuit device can be configured to determine a recommendation based on the determined sensor value and to provide it to the user.

In various embodiments, the recommendation can comprise at least one recommendation selected from the group of hair care product recommendations, hair color product recommendations, hair styling product recommendations, and hair treatment recommendations.

In various embodiments, the sensor can have at least one sensor selected from the group of air humidity sensors, temperature sensors, UV radiation sensors, hair thickness sensors, hair moisture sensors, damage level sensors, and hair and/or skin fat content sensors.

In various embodiments, the sensor can be optically and/or physically in contact with an exterior surface of the device body.

In various embodiments, the adhesive layer can have a pressure sensitive adhesive and/or a Velcro material.

In various embodiments, the electronic circuit device can have a wireless data interchange device.

In various embodiments, a hair examination system is provided. The hair examination system can have a hair analysing device according to various embodiments and a display device, wherein the at least one hair analysing device can be configured to transmit the sensor value and/or the recommendation to the display device by employing the data exchange device.

In various embodiments, the display device can have or can be a computer screen, a smartphone, a tablet, a smartwatch, an iPad, or a laptop.

In various embodiments, a method of providing hair condition information is provided. The method can have an arranging of a hair analysing device according to various embodiments on a user's hair, detecting at least one sensor value of the at least one sensor of the hair analysing device, and providing the at least one sensor value to the user.

In various embodiments, the method can further have determining at least one recommendation based on the at least one sensor value.

In various embodiments, the recommendation can comprise at least one recommendation selected from the group of hair care product recommendations, hair styling product recommendations, hair color product recommendations, and hair treatment recommendations.

In various embodiments, the method can further have triggering an order of a recommended hair care product, hair styling product and/or hair color product.

In various embodiments, arranging the hair analysing device can have an arranging for a long time period compared to a time period required for individually detecting the at least one sensor value, and detecting the at least one sensor value can be repeated or continuous during the time period.

In various embodiments, the method can further have transmitting the at least one sensor value to an external data processing device and receiving the at least one recommendation from the external data processing device, wherein determining the at least one recommendation can be done based on the at least one sensor value by employing the external data processing device.

In the following detailed description, reference is made to the accompanying drawings which form a part of the present application and in which is shown by way of illustration specific embodiments in which the present disclosure can be practiced. In this regard, directional terminology such as “top”, “bottom”, “front”, “back”, “front”, “rear”, etc. is used with reference to the orientation of the described figure(s). Since components of embodiments can be positioned in a number of different orientations, the directional terminology is illustrative and is in no way limiting. It should be understood that other embodiments can be utilized and structural or logical changes can be made without departing from the scope of the present disclosure. It should be understood that the features of the various embodiments described herein can be combined with each other unless specifically stated otherwise. The following detailed description is therefore not to be taken in a limiting sense, and the scope of the present disclosure is defined by the appended claims.

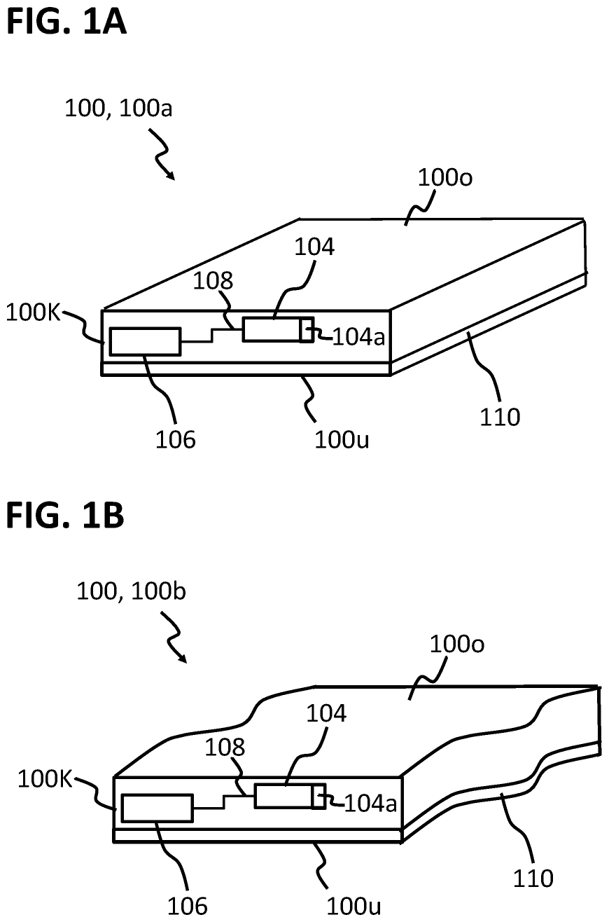

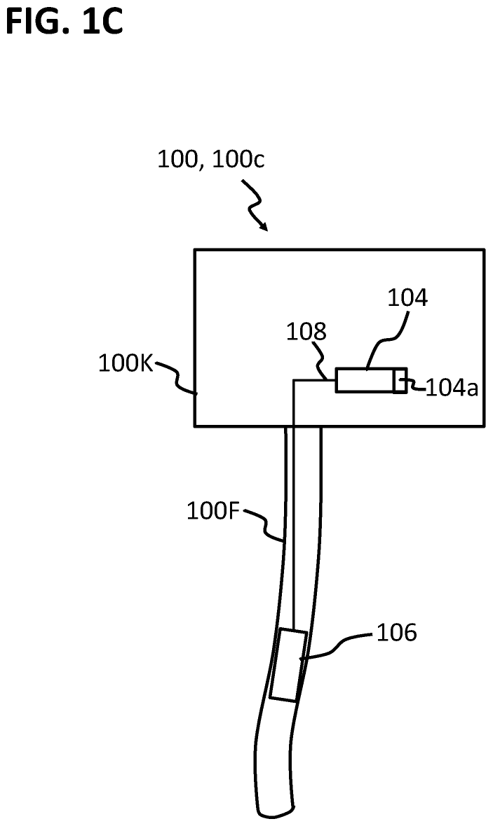

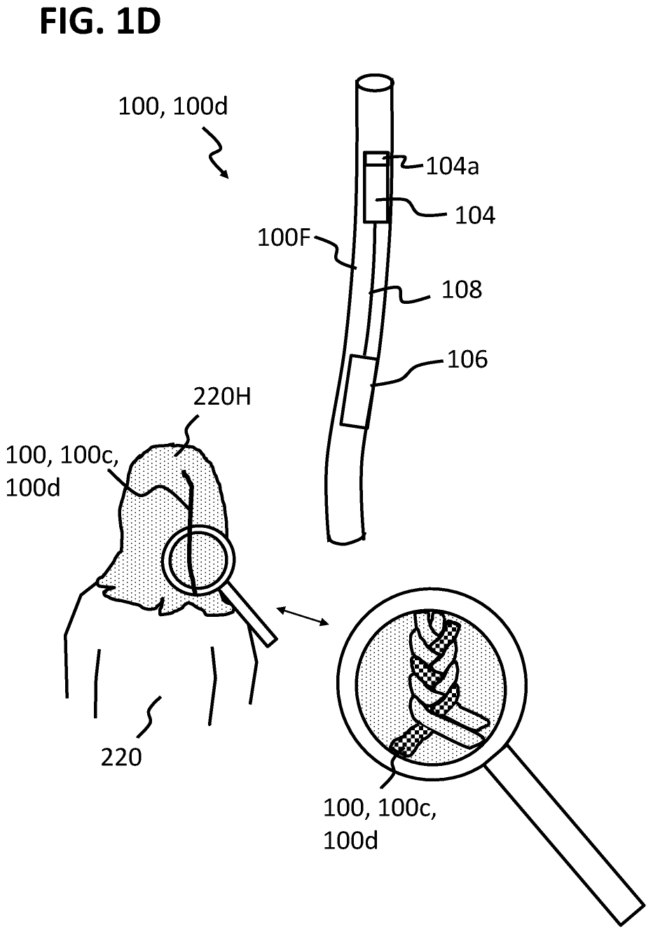

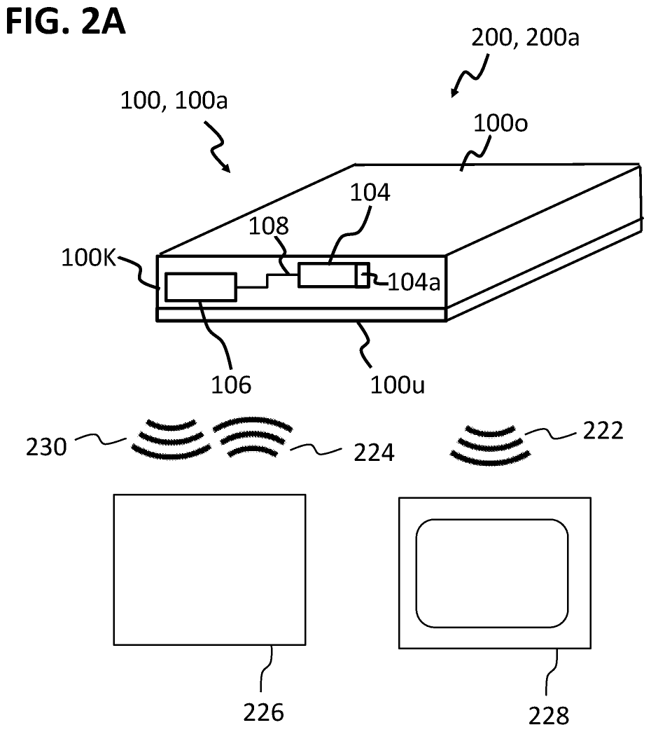

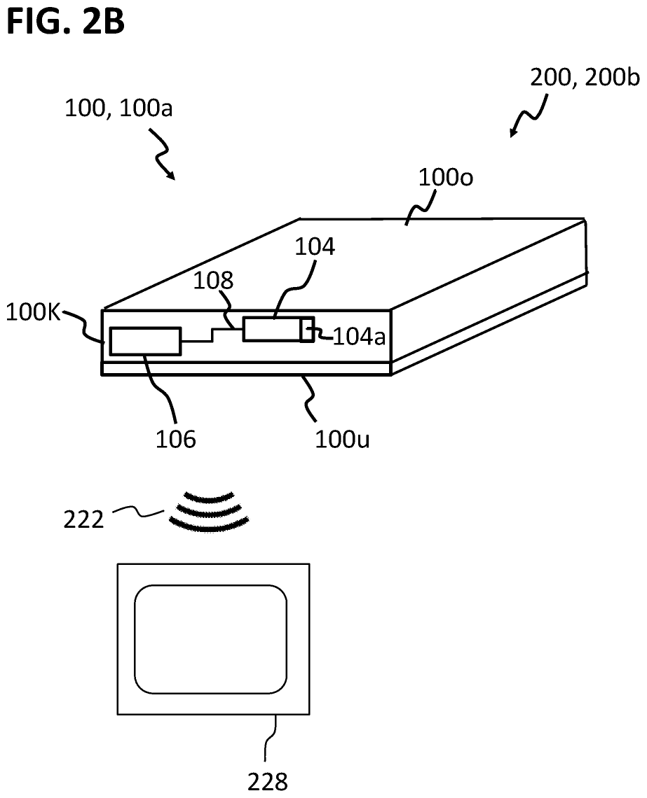

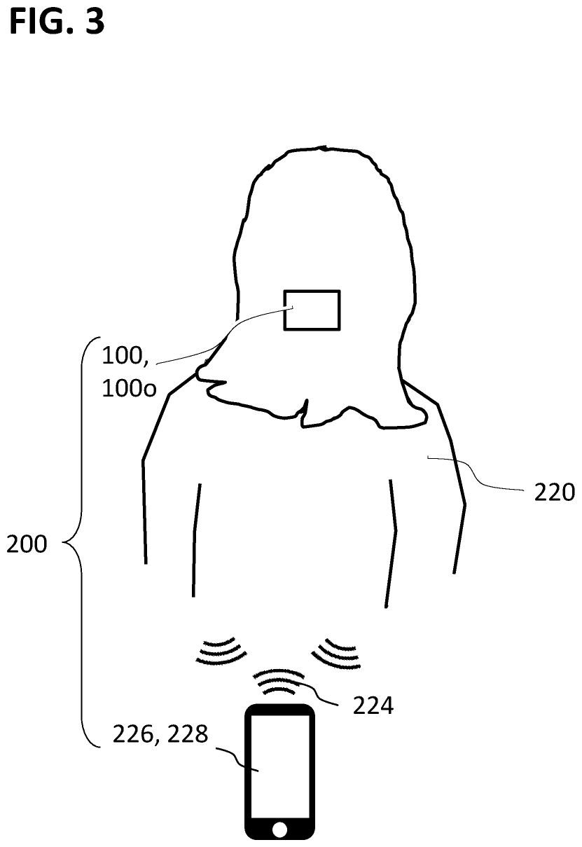

FIG. 1A to FIG. 1D are schematic representations of hair analysing devices 100 according to various embodiments, FIG. 2A shows a schematic representation of a hair examination system 200, 200a according to various embodiments in connection with an external data processing device 226, FIG. 2B shows a schematic representation of a hair examination system 200, 200b according to various embodiments, and FIG. 3 shows a schematic representation of an application of a hair examination system 200 according to various embodiments.

In various embodiments, a hair analysing device 100 is provided (various embodiments are identified as 100a, 100b, 100c, or 100d, respectively).

In various embodiments, the hair analysing device 100 can have a device body 100K. The device body 100K can be configured to be arrangeable in or on the hair 220H of a user 220.

In various embodiments (not shown), the device body 100K can be designed to be arrangeable in the hair 220H of the user 220 by employing its mechanical design and/or elastic properties, for example, as a hair tie, headband, decorative hair comb, hair clip, or the like. The device body 100K can be formed of or have a solid material such as plastic or metal.

As shown in FIG. 1A, the device body 100K can be provided on at least one side 100u on its surface with an adhesive layer 110 by which the hair analysing device 100 can be arrangeable in/on the hair of the user 220. The adhesive layer 110 can completely or partially cover the device body 100K on the side 100u.

In various embodiments, the hair analysing device 100 can be releasably fastened in or on the hair of a user by employing the adhesive layer 110. The adhesive layer 110 can have a pressure sensitive adhesive and/or a Velcro material in various embodiments.

As shown in FIG. 1B for the hair analysing device 100b with reference to the device body 100K shown in a wavy manner, it should be noted that the device body 100K can be flexible in various embodiments. This can make it possible to adapt the hair analysing device 100b to a head shape and/or a hairstyle of the user when arranging it in/on the hair 220H of the user 220. For example, the device body 100K can be formed as a film or a foil.

In various embodiments, an upper side 100o of the device body 100K can be decorative.

As shown in FIG. 1C and FIG. 1D, in various embodiments, the device body 100K can have a thread-shaped region 100F (FIG. 1C) or consist of such (FIG. 1D).

The thread-shaped region 100F may, in various embodiments, be designed such that, for example, in terms of its length, thickness and/or flexibility, it can be interlaced into the hair 220H of the user 220, as shown in FIG. 1D below. In various embodiments, the hair analysing device 100 can already be fastened or be fastenable to the hair 220H by interlacing the thread-shaped region 100F. In other embodiments, the hair analysing device 100 can also be fastened or be fastenable to the hair 220H by other fastening features, for example, by employing the adhesive layer 110, by employing a clamping device, by employing a comb-shaped part of the hair analysing device, or the like.

In various embodiments, the hair analysing device 100 can have at least one sensor 106 arranged in or on the device body 100K for detecting at least one sensor value.

The at least one sensor 106 can be used in various embodiments as described above for determining sensor values and for determining a recommendation.

In various embodiments, the at least one sensor 106 can be arranged in the device body 100K such that it can interact with the hair 220H and/or an environment of the hair 220H.

In various embodiments, in which the device body 100K has or consists of a thread-shaped region 100F, at least a part of the at least one sensor 106 can be arranged in the thread-shaped region 100F. For example, the part of the sensor 106 or the sensor 106 can be arranged in the thread-shaped region 106F such that the sensor measured value is determined at or near a location of the hair on which the sensor 106 or the part of the sensor 106 is located. In various embodiments, multiple (for example, similar or different) sensors 106 can be arranged in the thread-shaped region 106F, which can provide sensor values from different regions, for example, along a hair length.

In various embodiments, the at least one sensor 106 can have any, for example, also in principle known, detection device which is suitable to provide sensor data that can provide information about a hair condition of a user and/or an environment that the user's hair 220H 220 can be exposed, and which can be arrangeable due to miniaturization of such devices in a hair analysing device 100 as described herein (for example, in a hair analysing device formed as an adhesive film, hair clip, hair tie, headband, braid or the like).

In various embodiments, the at least one sensor 106 can have a hair thickness sensor as described above, for example, an optical sensor and/or an ultrasonic sensor and/or a laser.

In various embodiments, as described above, the at least one sensor 106 can have a spectrometer and/or a camera for detecting an absorption feature of a substance to be detected, for example, a lipid (for example, a lipid which can be part of a user's cutaneous sebum), a cysteic acid, water or the like.

In various embodiments, the at least one sensor 106, as described above, can have a thermometer for detecting a hair temperature, a scalp temperature, and/or an ambient temperature of the user.

In various embodiments, the at least one sensor 106 can have a camera, as described above, for example, as a hair color sensor and/or for determining a hair porosity.

In various embodiments, as described above, the at least one sensor 106 can have an air humidity sensor and/or a UV radiation sensor.

In various embodiments, the hair analysing device 100 can have an electronic circuit device 104 arranged in or on the device body 100K.

The electronic circuit device 104 can, in various embodiments, be coupled to the at least one sensor 106, for example, by employing a connection 108, for receiving the at least one sensor value.

In various embodiments, in which the hair analysing device consists of a thread-shaped region 100F, the electronic circuit device 104, as shown in FIG. 1D, can be arranged in the thread-shaped region 100F.

In various embodiments, in which the hair analysing device has of a thread-shaped region 100F, the electronic circuit device 104, as shown in FIG. 1C, can be arranged in the differently (for example, film-like) shaped region, or the electronic circuit device 104 can be arranged in the thread-shaped region 100F (not shown).

Further, in various embodiments, the electronic circuit device 104 can be configured to provide the received at least one sensor value to the user 220.

In various embodiments, the electronic circuit device 104 can be or have a data processing device, for example, it can be equipped with a memory and a processor, for example, a microprocessor, which can be configured, for example, by programming, to receive the sensor data and either provide it directly to the user, or to use the sensor data to provide the recommendation.

The electronic circuit device 104 can be coupled in various embodiments, for example, by employing a connection 108, to the at least one sensor 106. In the presence of a plurality of sensors 106, the circuit device 104 can have its own coupling to each of the sensors 106. The coupling can in various embodiments have or be an electrically conductive connection, a (glass) fiber connection and/or a wireless connection. The electronic circuit device 104 can be configured to receive the at least one sensor value from the at least one sensor 106.

The electronic circuit device 104 can be configured in various embodiments to determine at least one recommendation based on the received at least one sensor value and to provide it to a user 220.

The measured sensor values can, as described above, either be evaluated directly in the hair analysing device, for example, by employing the electronic circuit device, or indirectly evaluated by being transmitted to an external data processing device to be evaluated there, for example, as described above. In this case, the sensor data or parts of the sensor data can be evaluated in various embodiments in a manner known in principle, for example, by employing an image processing software, for example, to determine the hair color, the hair thickness, to determine the hair porosity or the like, and/or in comparison with (for example, empirically obtained) database entries.

In various embodiments, the determined hair thickness, hair color, hair porosity, hair/skin temperature, the determined hair damage level (for example, based on a cysteic acid concentration), the determined hair, scalp and/or ambient temperature or a temperature variation determined therefrom, the detected air humidity, UV radiation and/or one or more further directly determined sensor values and/or parameters determined on the basis of the sensor values are included in a determination of a recommendation, for example, a product or treatment recommendation.

In various embodiments, arranging the hair analysing device 100 can have arranging for a long time period as compared with a time period required for a single detection of the at least one sensor value. For example, the hair analysing device 100 can be left for a period of one or more hours, for example, a large part of the day.

In various embodiments, the detection of the at least one sensor value can be performed repeatedly or continuously during the time period in which the hair analysing device 100 is arranged in or on the hair 220H. In this way, it is possible, for example, to carry out the hair examination repeatedly or continuously during a day, for example, of a typical elapsing day, so that the sensor values characterizing the hair condition and/or external influences can be detected for a situation which, for example, regularly recurs. Accordingly, product and/or treatment recommendations can be adapted to a predictably reoccurring situation. For example, in the case of expected (for example, because of repeated occurrence) stay in rooms with low air humidity, for example, a moisturizing hair care product can be recommended, for example, in the expected (longer) stay outdoors, a hair care product with UV protection, etc.

As described above, in various embodiments, the electronic circuit device 104 can be configured to determine and provide to a user at least one recommendation based on the received at least one sensor value in which, as shown, for example, in FIG. 2B, the recommendation itself (directly) determines, for example, as described above, for example, by comparison with reference values, value ranges and/or databases stored in a memory of the electronic circuit device 104, wherein the reference values, value ranges or databases can each be assigned least one recommendation.

As described above, in various embodiments, the electronic circuit device 104 can be configured to determine and provide to a user at least one recommendation based on the received at least one sensor value in which, as shown, for example, in FIG. 2A, the recommendation indirectly determines, for example, as described above, for example, by transmitting (illustrated as transmission signal 230) the at least one sensor value to an external data processing device 226, for example, an external computer, for example, a cloud, comparing it to reference values, value ranges and/or databases, stored in a memory of the external data processing device 226, wherein the reference values, value ranges or databases can each be assigned at least one recommendation. The hair analysing device 100 can be configured to receive the recommendation determined by the external data processing device 226, that is, the determined recommendation can be transmitted to the hair analysing device 100 (illustrated as a transmission signal 224).

In various embodiments, a hair examination system 200 can be provided, as is shown in FIG. 2A, FIG. 2B and FIG. 3 by way of example. The hair examination system can have a hair analysing device 100 according to various embodiments and a display device 228.

For transmitting the at least one sensor value and/or the at least one recommendation to the display device 228 and/or for transmitting the at least one sensor value to the external data processing device 226 and/or for receiving the at least one recommendation from the external data processing device 226, the hair analysing device 100 can be equipped with a transmission device 104a for wireless data transmission, for example, with a chip or tag that allows wireless data transmission, for example, by employing Bluetooth, WLAN, Zigbee, NFC, Wibree, Threald, WiMAX or the like. In various embodiments, the transfer device 104a can be part of the electronic circuit device 104.

As shown in FIG. 3, in various embodiments, the display device 228 can form an integrated unit with the computing device 226, for example, in the form of a smartphone, tablet, laptop, smartwatches, iPads, or the like.

In various embodiments, the hair analysing device can have an integrated power supply (not shown), such as rechargeable batteries or the like, which can provide an operating voltage to the electronic circuit device 104, the transfer device 104a, and the at least one sensor 106. The power supply can be made interchangeable in various embodiments. In various embodiments, the power supply (for example, waterproof) can be arranged closed, for example, cast in, in the hair analysing device 100. In that case, for example, the power supply can be inductively rechargeable.

In various embodiments, the hair analysing device 100 can be considered in connection with the display device 228 and/or the external data processing device 226 as a hair examination system 200.

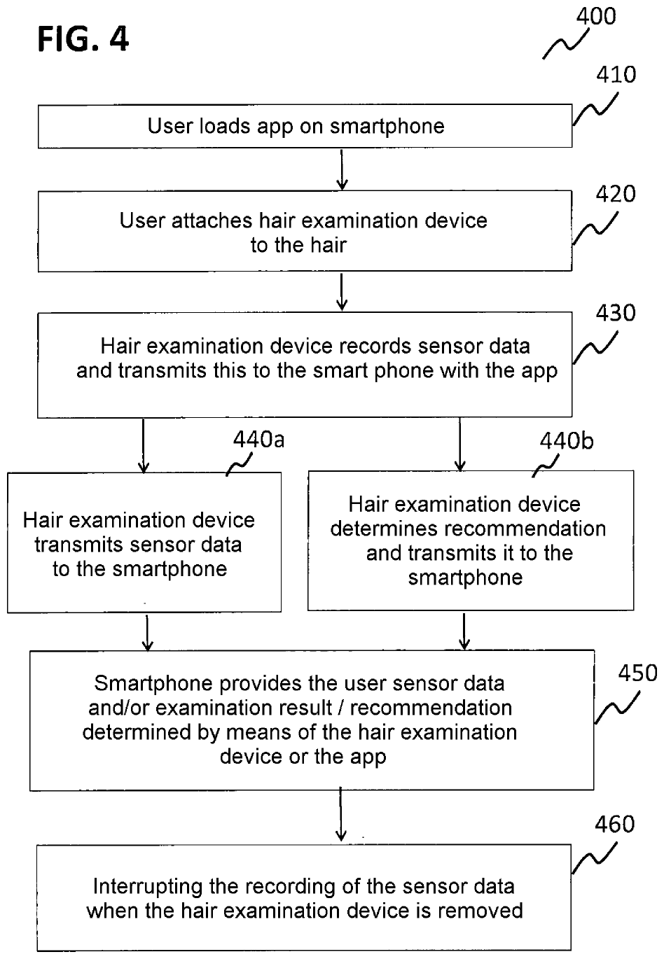

FIG. 4 shows a flowchart 400 of a method for providing hair condition information according to various embodiments.

A user can purchase a hair analysing device (a hairable) since he wants to know whether he is using the right care, coloring and/or styling products for his hair.

In various embodiments, the user can download a mobile application (app) belonging to the hairable to his smartphone (for example, via an app store) (in 410).

In various embodiments, it can be necessary and/or useful to register with his name and his e-mail address. In various embodiments, it can be necessary and/or useful for the user to store further information about himself, such as address or bank details.

In various embodiments, the user can attach the hair analysing device (the hairable), for example, in the form of an adhesive foil, in or on the hair (in 420).

In various embodiments, the user can wear the hairable during the day. During wear, the hair analysing device can record sensor data, for example, continuously (in 430).

The hair analysing device can, in various embodiments, analyze the external environment (UV radiation, air humidity, etc.) and the hair structure and scalp of the user. These data can be continuously synchronized with the app, that is, the examination results and/or recommendations determined by employing the hair analysing device can be transmitted to the smartphone (in 440b).

In other embodiments, the hair analysing device can transmit the sensor data to the smartphone (in 440a), and the smartphone can determine the exam results and/or recommendations (in 450). In various embodiments, the smartphone can provide the user with the sensor data and/or the examination result or the at least one recommendation determined by employing the hair analysing device or the app or the at least one recommendation (in 450).

At the end of an examination period, for example, in the evening, the user can remove the hairable from the hair, and the data recording is interrupted (in 460).

In various embodiments, enough data could already be collected so that the user receives an analysis via the smartphone app, which informs him about possible negative external influences and his hair condition.

Based on this data, the app can also give a recommendation for care, coloring and styling products, and/or use instructions (in 450).

In various embodiments, it is not necessary for the wearing of the hairable to occur on a daily basis, but data analysis and recommendations can improve as more data is collected.

In various embodiments, the recommendation for care, coloring and styling products and/or the use instructions can refer to a chemical composition of the recommended and not recommended care, coloring and styling products, for example, in the form of so-called DOs (recommendations) and DON'Ts (warnings). An example of this would be “Take or do not take a waxy product”.

Depending on the hair thickness and hair density, the user can be given a recommendation on a suitable styling product in various embodiments. For example, it can be disadvantageous to use a styling wax on thin, light hair, as this will quickly cause greasiness and unkemptness in such a hair type. The category “wax” includes products which are distinguished by a high proportion of wax components, vaselines, emulsifiers and oils or oily or oil-containing components. Also not recommended for thin light hair can be emulsions, which can also be exemplified by a high proportion of just described raw materials.

Depending on the curl condition (curls or rather waves), the user can be advised, in various embodiments, that a product either supports his natural curl image, or in the case of waves also strengthens (also known as pushing) and stabilizes. Certain polymers, such as Styleze CC10 (VP/DMAPA Acrylates Copolymer), Mirustyle CP (Polyquaternium-72), can be particularly helpful here. However, standard polymers, such as PVP, PVP/VA, etc. can well be suitable for holding the curls, and/or further polymers whose curl-holding or generation properties are known to those skilled in the art at the time of publication.

Curls usually require a lot of care, so that even with curly hair, a nourishing emulsion/lotion can be beneficial. Furthermore mousses can be applied to support bounce and springiness and to define the curls. In various embodiments, the polymers mentioned above for curl definition can be used. The curl definition can also be achieved with a light wax, whereas a (heavy) wax acts more as a drag on waves and probably can not help the consumer to maintain his styling result over a longer time period. However, in various embodiments, additional support can also be provided here by polymers (for example, as mentioned above) in order to prolong the result and the holding power.

Even with long hair, an application of a wax or gel can be unusual in various embodiments. In this case, the user can be advised to fall back to lighter consistencies, such as blow-dry sprays, mousses, lotions or the like, in order to support their look. Particularly sprayable or low-viscosity pumpable products can be well suited here, since they can be distributed particularly easily over the entire hair length. Depending on the desired styling effect, there can also be polymer types that can be used (for example, PVP, PVP/VA, Chitosan, Polyquaternium types, etc.).

In various embodiments, a choice of the right product can usually be related to the desired styling result. Thus, for example, a blow-dry spray or a mousse can be provided to aid in blow-drying, and can also contain nourishing styling components that allow working with comb and brush in wet hair (for example, Polyquaternium-4,11,46, etc., Chitosan, Polyacrylamidopropyltrimonium Chlorides, etc.).

A hairspray can be provided to fix the final hairstyle and also to protect against external influences endangering the hairstyle. An example of such an external influence would be the influence of high air humidity. Certain polymers can counteract this, for example, amphomer.

Short, strong hair can be well shaped and kept in shape with gels, waxes, pastes, creams and hair sprays.

The longer the hair, the stronger the desire of the user to remove the styling product from the hair before going to bed. Short hair which has been styled with waxes, pastes, creams, gels or the like can usually wash out quickly with water and dry quickly afterwards (possibly by using a hair dryer). Longer hair styled with a styling mousse and/or fixed with a hairspray is often brushed off. For this purpose, it can be helpful when the applied fixing polymer is easily brittle and fragile and can be removed, for example, by using a comb/a brush. Said polymers would be, for example, PVP, PVP/VA, amphomer, etc.

Users with African-American hair can prefer products that have one thing in common: much care. This target group can feel that their hair is very dry and brittle and needs much moisture. In particular, rich, creamy textures can be desirable here, and also oil sprays that make the hairstyle of the user look healthy and well-groomed. Conditioning polymers can help keep the hair in shape without making it stiff and inflexible. Rich waxes and emulsions can provide additional care to the hair and can easily weigh down the hair, so that the hair does not fly (anti-frizz). What would thus be a “no go” on European hair, can not be nurturing/weighing/greasy enough for African-American hair.

Users with damaged, for example, bleached, hair can need more care in their products than people with healthy (“virgin”) hair.

In various embodiments, in addition to the chemical properties or instead physical properties of the recommended and not recommended care and styling products (DOs and DON'Ts, for example, viscosities, evaporation properties, stickiness) can be crucial or influential as to whether a product is considered as recommended or not for a given hair type.

The longer the hair, the lower the stickiness of formulations should be. Users with long hair especially often want a styling product that holds and styles, but does not stick very hard. Many of them can prefer a natural hair feel and appearance.

In addition to mousses and light sprays or emulsions for blow-drying, long-haired women can often use hairspray to fix their hairstyle. Again, a non-strong adhesive product can be preferred. In addition, however, it can be desirable for the hairspray to be finely dispersed on the hair to avoid the said effect (a concreted/unnatural looking hairstyle).

Even users with curls can care about supporting the curls and making them look natural, and not cement their look. The thicker and shorter the hair, the higher the product viscosity can be, since it often helps to shape the hair when styling. Here, as already described above, pastes, creams, gels with a high viscosity are suitable.

In various embodiments, in addition to the chemical properties and/or the physical properties, a making-up (also referred to as application form, etc.) of the recommended and not recommended care and styling products (DOs and DON'Ts, for example, spray rather than gel) can be decisive or influential on whether or not a product is considered to be recommended for a given hair type. This is partly described above.

In various embodiments, the determined recommendation can be used to make an optimal/personalized hair treatment product, for example, a styling product, for example, as an order to make an optimal/personalized hair treatment product, for example, a styling product.

This can be initiated, for example, by calling a manufacturer's website for optimal/personalized hair treatment products, such as styling products.

The optimal/personalized hair treatment product can be a product specially made for the customer or a so-called mass customized product. In the case of a “mass customized” product, an individualization can be achieved by varying a few characteristics of a product that are decisive from the customer's point of view. Preferably, these “mass customized” products are based on the concept of modularization, that is, the product can be assembled individually from various modules/building blocks.

Often, there are many dependencies between the many different features/ingredients of a product, which can be expressed as “commandments” or “prohibitions.” In order to obtain a clear product definition, it can be advantageous for the ordering process to proceed with the aid of a product configurator. This configurator helps the customer to select the characteristics/ingredients and draws attention to the permitted/inadmissible combinations of features, wherein the latter then can not be selected.

With the help of a product configurator, for example, the selection of chemically and/or physically incompatible ingredients or the selection of the ingredients unsuitable for the determined hair condition can be avoided. Conversely, the selection of suitable ingredients for the determined hair condition can be predetermined or suggested by the product configurator.

In various alternative embodiments, a user input can be utilized on a delivery device for the optimized delivery of hair treatment product. This delivery device can preferably be present at a hairdresser or at a point of sale (POS) of hair treatment agents. The user can select his hair condition and, if necessary, the desired amount of product using a digital display and a touchscreen. For this purpose, a stored recommendation can already be programmed in the device, for example, stored in a database. Such recommendations can be known from leaflets of hair colorations, on which it can frequently be noted that, for example, two coloration packages should be used for shoulder-length hair. A (quantity and) product recommendation can be made by employing a previous individual input of the hair length of the user. This can either be confirmed, extended or reduced by the user when, for example, he already knows that he tends to use more/less product than is normally stated on packaging.

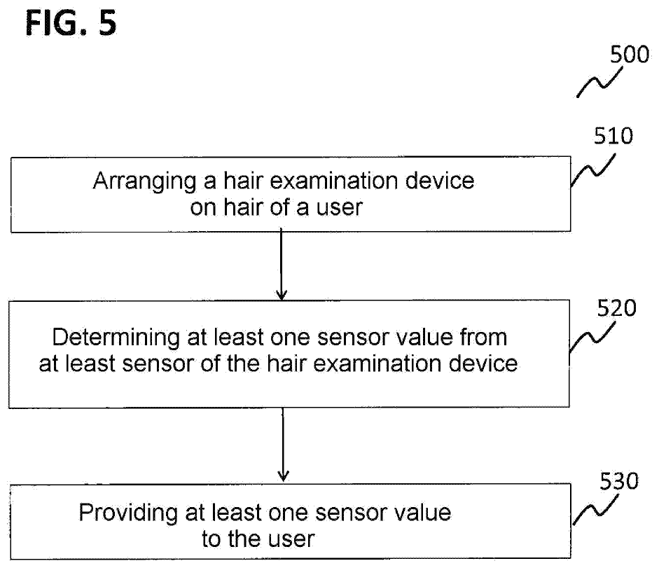

FIG. 5 shows a flowchart 500 of a method for providing hair condition information according to various embodiments.

A hair analysing device according to various embodiments and/or a hair examination system according to various embodiments can be used in a method of providing hair condition information according to various embodiments.

The method can have arranging a hair analysing device on hair of a user (for example, the hair analysing device according to various embodiments that, for example, can be part of the hair examination system according to various embodiments) (in 510), detecting at least one sensor value of at least one sensor of the hair analysing device (in 520) and providing the at least one sensor value to the user (in 530).

Programming, for example, software, can be used in various embodiments for the determinations described above. In this case, any software that provides a functionality described above can be used. In various embodiments, for example, in a case that a smartphone, a tablet or the like is used to carry out the method for providing hair condition information according to various embodiments, the software can be provided as an app.

In various embodiments, for example, by comparing the sensor values with provided reference values, it is possible, for example, to determine a suitable hair care agent, hair styling agent, a hair treatment recommendation or the like for the hair.

In various embodiments, the circuit device integrated into the hair analysing device and/or an external data processing device, for example, a smartphone, a tablet, a laptop, a smart mirror, a smartwatch, an iPad, or the like, can be suitable in order to be used when carrying out the method of providing hair condition information, for example, in determination operations, for example, by comparing with a database/reference values or the like. In various embodiments, the programming/software does not need to be provided on the smartphone, tablet, laptop, etc. For example, it can be sufficient when the circuit device integrated into the hair analysing device and/or the smartphone or the like is connected through the Internet, by employing WLAN or in another common way to a (for example, a further) external data processing device, for example, a computer, for example a cloud. In such a case, the calculations can be carried out, for example, by employing the (further) external data processing device, for example, by employing the computer, and the result can be provided to the smartphone/tablet or the like and/or the internal circuit device.

Further advantageous embodiments of the method are apparent from the description of the device and vice versa.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair analysing device, comprising:
a device body comprising an adhesive layer on a surface of at least one side and which is configured to be releasably fastened by employing the adhesive layer in or on the hair of a user;
at least one sensor arranged in or on the device body for detecting at least one sensor value; and
an electronic circuit device arranged in or on the device body,
the electronic circuit device being coupled to the at least one sensor for receiving the at least one sensor value, and
the electronic circuit device being configured to provide a received at least one sensor value to the user.

2. The hair analysing device, comprising:
a device body configured to be arranged in or on the hair of a user, wherein the device body is thread-shaped or has a thread-shaped region;
at least one sensor for detecting at least one sensor value, wherein the sensor or at least one of the sensors is arranged in the thread-shaped device body or the thread-shaped region of the device body; and
an electronic circuit device arranged in or on the device body,
wherein the electronic circuit device is coupled to the at least one sensor for receiving the at least one sensor value; and
the electronic circuit device being configured to provide a received at least one sensor value to the user.

3. The hair analysing device according to claim 1,
wherein the electronic circuit device is configured to determine a recommendation based on the determined sensor value and to provide the recommendation to the user.

4. The hair analysing device according to claim 3, wherein the recommendation comprises at least one recommendation selected from the group of hair care product recommendations, hair color product recommendations, hair styling product recommendations, or hair treatment recommendations.

5. The hair analysing device according to claim 1,
wherein the sensor has at least one sensor selected from the group of air humidity sensors, temperature sensors, UV radiation sensors, hair thickness sensors, hair moisture sensors, damage level sensors, or hair and/or skin fat content sensors.

6. The hair analysing device according to claim 1,
wherein the sensor is optically and/or physically in contact with an outer surface of the device body.

7. The hair analysing device according to claim 1,
wherein the adhesive layer has a pressure-sensitive adhesive and/or a Velcro material.

8. The hair analysing device according to claim 1,
wherein the electronic circuit device has a wireless data exchange device.

9. A hair examination system, comprising:
a hair analysing device according to claim 8; and
a display device,
the at least one hair analysing device being configured to transmit to the display device the sensor value and/or the recommendation by employing the data exchange device.

10. The hair examination system according to claim 9,
wherein the display device comprises a computer screen, a smartphone, a tablet, a smartwatch, an iPad or a laptop.

11. A method of providing hair condition information, comprising:
arranging a hair analysing device according to claim 1 on hair of a user;
detecting at least one sensor value of the at least one sensor of the hair analysing device; and
providing the at least one sensor value to the user.

12. The method of claim 11, further comprising:
determining at least one recommendation based on the at least one sensor value.

13. The method according to claim 12,
wherein the recommendation comprises at least one recommendation selected from the group of hair care product recommendations, hair styling product recommendations or hair treatment recommendations.

14. The method according to claim 11,
wherein the arranging of the hair analysing device comprises arranging for a long time period compared to a time period required for a single detection of the at least one sensor value, and
wherein detecting the at least one sensor value is performed repeatedly or continuously during the time period.

15. The method of claim 11, further comprising:
transmitting the at least one sensor value to an external data processing device; and
receiving the at least one recommendation from the external data processing device,
wherein determining the at least one recommendation is done based on the at least one sensor value by employing the external data processing device.

16. The hair analysing device according to claim 2,
wherein the electronic circuit device is configured to determine a recommendation based on the determined sensor value and to provide the recommendation to the user.

17. The hair analysing device according to claim 16, wherein the recommendation comprises at least one recommendation selected from the group of hair care product recommendations, hair color product recommendations, hair styling product recommendations, or hair treatment recommendations.

18. The hair analysing device according to claim 2,
wherein the sensor has at least one sensor selected from the group of air humidity sensors, temperature sensors, UV radiation sensors, hair thickness sensors, hair moisture sensors, damage level sensors, or hair and/or skin fat content sensors.

19. The hair analysing device according to claim 2,
wherein the sensor is optically and/or physically in contact with an outer surface of the device body.

20. The hair analysing device according to claim 2,
wherein the adhesive layer has a pressure-sensitive adhesive and/or a Velcro material.
